(19) United States
(12) Patent Application Publication
(10) Pub. No.: US 2025/0249455 A1
(43) Pub. Date: Aug. 7, 2025

(54) A MICROFLUIDIC STRUCURE FOR DIFFERENTIAL EXTRACTION

(71) Applicant: University of Virginia Patent Foundation, Charlottesville, VA (US)

(72) Inventors: Michael Shane Woolf, Midlothian, VA (US); James P. Landers, Charlottesville, VA (US); Larissa L. Cunha, Charlottesville, VA (US)

(21) Appl. No.: 18/701,104

(22) PCT Filed: Oct. 12, 2022
(86) PCT No.: PCT/US2022/077977

§ 371 Date: Apr. 12, 2024

Related U.S. Application Data
(60) Provisional application No. 63/262,438, filed on Oct. 12, 2021.

(51) Int. Cl. B01L 3/00; C12N 15/10

(52) U.S. Cl. CPC B01L 3/502761; B01L 3/502715; B01L 3/50273; B01L 3/502738; C12N 15/1003; B01L 2200/0621; B01L 2200/0647; B01L 2200/0684; B01L 2200/0689; B01L 2200/16; B01L 2300/0803; B01L 2400/0409; B01L 2400/0677

(57) ABSTRACT

A differential extraction device includes a first fluidic layer, a second fluidic layer, and a valving layer disposed between the first and second fluidic layers and include multiple chambers and microfluidic channels. The valving layer provides the ability to selectively allow and prevent flow between various chambers. Reagent chambers deliver reagent to a sample chamber and multiple recovery chambers receive material from the sample chamber. The valving layer provides the ability to selectively allow and prevent flow between various chambers.

### PRIORITY APPLICATION

This application claims priority to U.S. Provisional Patent Application Ser. No. 63/262,438, filed on Oct. 12, 2021, the disclosure of which is incorporated by reference herein in its entirety.

### BACKGROUND

Sexual Assault Evidence Collection Kits (SAECKs) are one of the most common types of evidence received in forensic laboratories and are used to collect biological evidence from victims of sexual assault and rape. The unique nature of SAECK DNA evidence contributes to backlogs and slow analysis turnaround times, resulting in an ever-growing demand for forensic DNA services. The goal in SAECK processing is preferential cellular lysis and separation that yields discrete fractions, namely the non-sperm fraction (NSF) and sperm (SF) fractions, and downstream DNA profiling.

The conventional method for SAECK processing is based on a multistep, multi-tube process that includes differential extraction (DE), a laborious and time-consuming process that often fails to generate adequate sperm cell DNA recovery. The process consists of combining a sample cutting that contains sperm and epithelial cells (e-cells) with an enzymatic lysis cocktail that preferentially lyses e-cells. An incubation step is used to activate the enzyme. Centrifugation is used to pellet sperm cells to the bottom of a tube. A supernatant, the liquid above the pellet sperm cells, following this step comprises the NSF. The SF is then generated by adding a sperm cell lysis cocktail to liberate sperm DNA.

The multistep, multitube process requires long incubation times, several manual tube transfers, and a centrifugation of five minutes or more. A total process workflow time exceeds four hours. The process involves manually intensive workflows and often fails to generate adequate sperm cell DNA recovery.

Because timely processing of SAECKs is crucial in ensuring the judicious prosecution of sexual predators from the population, alternative methods towards the processing of SAECKs have been investigated to address the backlog challenge associated with traditional DE. Mechanized robotic platforms have been reported to reduce hands-on time, however these require considerable financial investment and offer no significant changes to the chemistry and procedure itself, and no enhancement of sperm cell DNA recovery.

### SUMMARY

A differential extraction device includes a first fluidic layer, a second fluidic layer, and a valving layer disposed between the first and second fluidic layers and include multiple chambers and microfluidic channels. The valving layer provides the ability to selectively allow and prevent flow between various chambers. Reagent chambers deliver reagent to a sample chamber and multiple recovery chambers receive material from the sample chamber. The valving layer provides the ability to selectively allow and prevent flow between various chambers.

In one example, the valving layer includes vaporizable and meltable portions to respectively open and close valves, and may be operated by laser, heat source, acoustic actuation, electrical actuation, or other actuation. Fluid flow may be provided by selectively spinning the layers of the device after valves are actuated, or by the use of gravity, differential pressure, pumps, capillary action, vibration, or otherwise.

In one example, the device is a valved microfluidic device that provides for automated preparation of Sexual Assault Evidence Collection Kits (SAECKs). The device may provide one or more domains capable of individually processing single evidence cuttings via a stepwise e-cell lysis, three intermediate wash steps, and a final sperm cell lysis. Reagent chambers hold reagents and fluidic control is provided for the successful fractionation of NSF and SF, achieved by the incorporation of active valving methods for channel opening and closure.

### BRIEF DESCRIPTION OF THE DRAWINGS

FIG. 1 is an exploded view of an example microfluidic assembly according to an example embodiment.

FIG. 2 is a block diagram side view of an example microfluidic processing system according to an example embodiment.

FIG. 3 is a top view block diagram of the microfluidic assembly of FIG. 1 according to an example embodiment.

FIGS. 4A, 4B, 4C, 4D, 4E, 4F, FIGS. 4G and 4H illustrate process workflow generally at for performing differential extraction of DNA according to an example embodiment.

FIGS. 4I and 4J illustrate an alternative valve closing structure according to an example embodiment.

FIG. 5 is a block diagram of a computer system for controlling processes performed on a microfluidic assembly according to an example embodiment.

### DETAILED DESCRIPTION

In the following description, reference is made to the accompanying drawings that form a part hereof, and in which is shown by way of illustration specific embodiments which may be practiced. These embodiments are described in sufficient detail to enable those skilled in the art to practice the invention, and it is to be understood that other embodiments may be utilized and that structural, logical and electrical changes may be made without departing from the scope of the present invention. The following description of example embodiments is, therefore, not to be taken in a limited sense, and the scope of the present invention is defined by the appended claims.

Biological evidence originating from victims of sexual assault is often comprised of unbalanced cellular mixtures with significantly higher contributions from the victim's genetic material. Enrichment of the forensically-critical sperm fraction (SF) with single-source male DNA currently relies on differential extraction (DE), a manually-intensive process that is prone to contamination. Due to DNA losses from sequential washing steps, some existing DE methods often fail to generate sufficient sperm cell DNA recovery for perpetrator(s) identification.

A valved microfluidic device provides for automated preparation of Sexual Assault Evidence Collection Kits (SAECKs). The device may provide one or more domains capable of individually processing single evidence cuttings via a stepwise e-cell lysis, three intermediate wash steps, and a final sperm cell lysis. Reagent chambers hold reagents and fluidic control is provided for the successful fractionation of NSF (non sperm fraction) and SF, achieved by the incorporation of active valving methods for channel opening and closure to perform differential extraction.

In one example, a laser actuates valves of a microfluidic device to automate SAECKs processing by providing timed reagent release and temperature control for sequential enzymatic reactions. The device may include a stack of discs with laser actuatable valves that eliminate the need for external hardware. The stack of disks may be rotated to provide centrifugal force for fluid flow control in response to valve control operations. The microfluidic device may be used to avoid large and costly robotic platforms for automation of differential extraction.

In one example, the microfluidic device mimics a conventional differential extraction (DE) workflow with features such as a pipetting, metering, fractionation, and centrifugation. A well-established print-cut-laminate (PCL) method of disc fabrication may be used to integrate the DE unit operations into a multi-layer disc structure. This can be accomplished by using common office equipment and overhead transparency sheets. Typically, the discs consist of two polyethylene terephthalate (PeT) layers, and 2 fluidic layers separated by a valving layer. The layers may be heat-bonded with a laminator. Polymethyl methacrylate (PMMA) accessory pieces may be used to give the channels height for increased volume.

Other bio-compatible materials may be used to form a suitable microfluidic structure in further examples by means of injection molding, 3-D printing or other process. The valves may be formed of material that is meltable via a laser or other heat producing mechanism such as resistive heating elements, or may be mechanically or electromechanically controllable valves. While force may be provided by rotating the microfluidic structure move fluids between chambers and separate materials within chambers, other forces may be used such as differences in pressure, vibration, filtration, or gravity. Syringe pumps may be used to provide a suitable difference in pressure to move fluids in a desired manner between chambers in some examples. Vibration sources, acoustic energy, and/or capillary action may be used to move fluids in further embodiments.

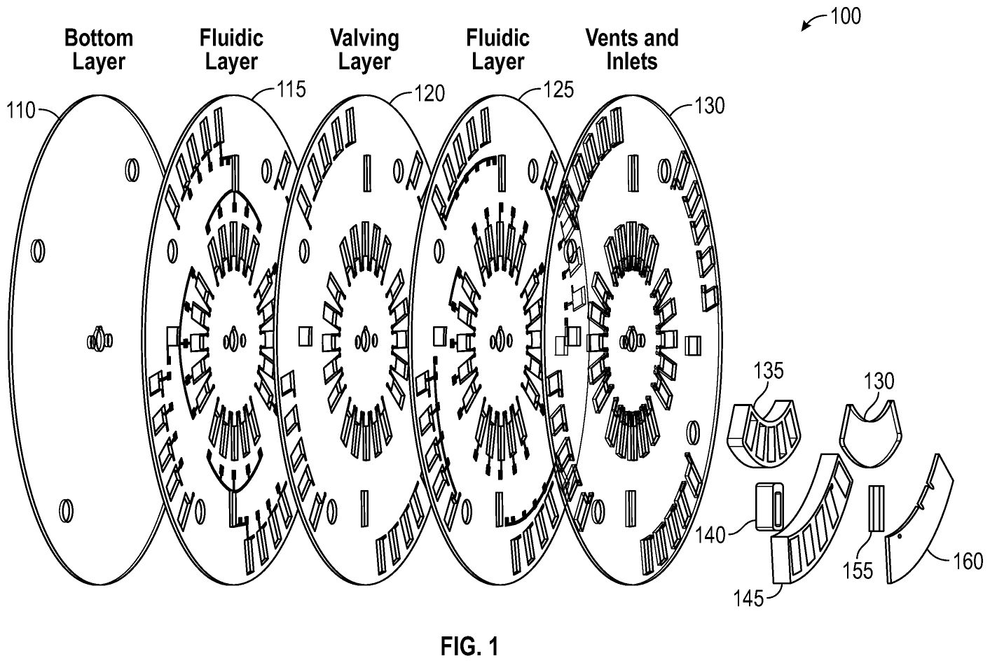

FIG. 1 is an exploded view of an example microfluidic assembly 100. Assembly 100 includes a bottom base PeT layer 110, a first fluidic layer 115 bound to the base layer 110 with heat sensitive adhesive (HSA). A primary black or dark PeT valving layer 120 is disposed above and bound to the first fluidic layer 115. A second fluidic layer 125 is disposed above and bound to the valving layer 120. A top PeT layer 130, a vents and inlets layer, is disposed above and bound to the second fluidic layer.

Accessory pieces 135, 140, 145 may be added to the top PeT layer 130 with corresponding openings to the second fluidic layer 125 to provide channel depth. The accessory pieces may be formed of PMMA and may be used to expand a volume for reagents used in processing a sample as well as providing sufficient volume for inserting a sample via accessory piece 140. Coverlets 150, 155, and 160 may also be added to the accessory pieces 135, 140, and 145. The coverlets 150, 155, 160 may be formed of PMMA and PeT or other bio-compatible material and may be added post-lamination with pressure sensitive adhesive (PSA).

Assembly 100 in one example has five laminated layers of PeT transparency film. The base layer 110 and top layer 130 may consist entirely of PeT, while the fluidic layers 115 and 125 serve as the primary microfluidic layers bound by heat-sensitive adhesive (HSA), which is applied prior to device assembly. Valving 120 functions as a primary valving layer and is precoated with two layers of toner on each side. Microfluidic inlets, vents, and voids are cut into the appropriate layers via laser ablation such as via a simple CO₂ laser. PMMA was used for the accessory pieces 135, 140 and 145 to add chamber depth and volume and was attached post-lamination with pressure-sensitive adhesive (PSA).

Each layer of assembly 100 may be a disc, which in one example have a diameter of about 14.7 cm. Assembly 100 may be a PCL device comprised of five layers of polyethylene terephthalate films (PeT) and polymethyl methacrylate (PMMA) accessory pieces. Heat and pressure sensitive adhesives (HSA and PSA, respectively) may be used to facilitate strong layer adhesion. The outer layers may be a clear PeT film (101.6 μm, Film Source, Inc. Maryland Heights, MO, USA). The primary fluidic layers may be clear PeT film bound on both sides by HSA (50.8 μm, EL-7970-39, Adhesives Research, Inc. Glen Rock, PA, U.S.A.). The valve layer may be a black PeT monolayer (bPeT, 75 μm, Lumirror* X30, Toray Industries, Inc. Chuo-ku, Tokyo, Japan), and functions as the laser valving medium. Prior to alignment and lamination, microfluidic features (e.g., inlets, vents, and chamber voids) may be cut into the appropriate layers via laser ablation (VLS3.50, Universal® Laser Systems, Scottsdale, AZ, U.S.A.). The laser ablated layers are aligned and lamination-bonded at 180-200° C. (13″ UltraLam 250B, Akiles Products, Inc. Mira Loma, CA, U.S.A.). PMMA accessory pieces with associated PeT coverlets (1.5 mm thick, McMaster-Carr, Atlanta, GA) may be used to add chamber depth and volume where needed; accessory pieces are attached to the top PeT layer of a laminated 5-layer disc with PSA (55.8 μm, ARcare 7876, Adhesives Research, Inc., U.S.A.).

When using other bio compatible materials for the layers, processes for forming fluidic structures that are suitable for such materials, such as etching laser, or other means for forming fluidic structures in the layers, may be used.

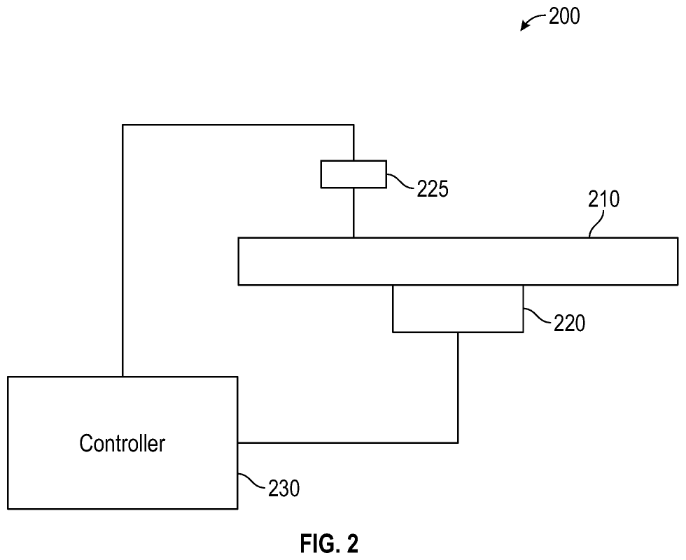

FIG. 2 is a block diagram side view of an example microfluidic processing system 200. For successful DE operations on the system 100, the discs 110, 115, 120, 125, and 130 shown in block form at 210 are mounted coaxially on a motor 220 to rotate the discs to create suitable centrifugal forces for centrifugation and assist in component separation and fluid movement in and between the fluidic layers. A laser 225, such as a laser diode, is mounted to controllable direct laser light orthogonally the disk assembly 210 to actuate valves on the valving layer 120 for controllable opening the valves and closing selected channels. The light from laser 225 passes through transparent upper layers to heat a flowable material in the valving layer 120 that either opens valves or closes channels. The laser diode may be used for microvalve opening and channel closure by alternatively vaporizing and melting the flowable material. The flowable material may be the PeT of the valving layer 120. The dark or black toner coating facilitates heating of the PeT layer.

Fluid flow control and laser control is provided via a controller 230 to perform a timed, stepwise reagent release, delivery, and recovery into the pertinent chambers to effect DE processing of a sample. A specific example of such control is provided below.

Valve opening enables timed reagent release and flow, promoted by centrifugation from disc rotation via motor 220, and prevents early access of fluids to downstream processes within the workflow. Similarly, channel closure events prevent issues such as backflow of fluid, avoiding undesirable mixing of reagents and recovered fractions. The laser-valving strategy also facilitates fractionation of the desired NSF and SF while circumventing capillary forces that prompt wicking and backflow.

In one example, the assembly 100 is a self-contained, single-use, assembly used for differential extraction, mirroring conventional DE workflow. Assembly 100 may provide one or more benefits that include reduction of (1) sample size and reagent volumes, (2) the number of manual sample handling and transfer steps, (3) cross-contamination risk, (4) potential sample loss, and (5) processing time. The assembly 100 utilizes rotational forces as the primary driving force to create pseudo-gravitational-centrifugal force to move fluids within the assembly 100. The use of centrifugal force inherently brings reduced instrument size and cost by removing the need for peripheral hardware such as tubing, pneumatic and hydraulic syringe pumps, and other equipment, thus avoiding the need for large, clunky, and costly robotic platforms for automation.

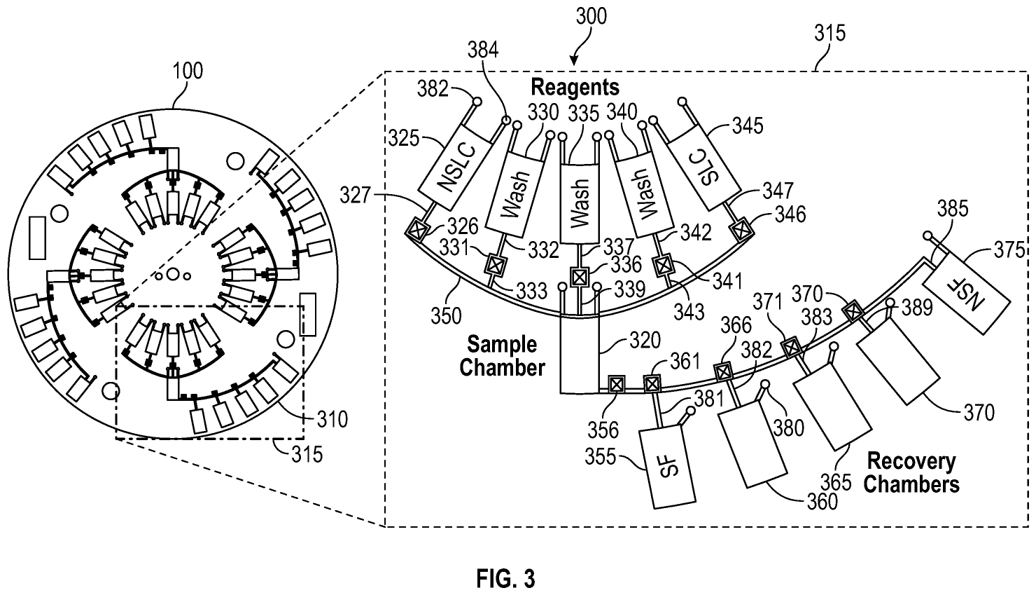

FIG. 3 is a top view block diagram 300 of the microfluidic assembly 100 may include multiple identical domains, one of four of which is indicated at 310 and shown in expanded detail at 315. Each domain is capable of processing a single evidence cutting containing sperm and non-sperm DNA. Each domain in one example is identical in structure and position, and equally arcuately spaced to minimize vibration of the assembly 100 during rotation. In one example, multiple cuttings collected from a single case may be processed in parallel in the domains. The processing is described in further detail below, but generally includes a stepwise e-cell lysis, three intermediate wash steps, and a final sperm cell lysis. The assembly may be modified for performing other processes in further examples.

Domain 310 includes multiple chambers coupled by microfluidic channels and valves disposed on different layers of the assembly 100. In one example, a sample chamber 320 is formed through layers 115, 120, 125, and 130. The size of the sample chamber 320 is expandable via accessory piece 140. The evidence cutting may be placed in this sample chamber 320. The sample chamber 320 extends through the valving layer 120 and also includes the fluidic layer 115.

Four reagent chambers 325, 330, 335, 340, and 345 are formed through layers 115, 120, 125, and 130 and may be fluidically coupled via a channel 350 formed on fluidic layer 115 to the sample chamber 320. Chamber 325 is used for a non-sperm lysis cocktail (NSLC). Chambers 330, 335, and 340 are used for wash fluids. Chamber 345 is used for a sperm lysis cocktail (SLC). The reagent chambers are also in the fluidic layer 125 and expandable via accessory piece 135. Recovery chambers 355, 360, 365, 370, and 375 are formed through layers 115, 120, 125, and 130 and are coupled via a channel 380 on fluidic layer 125 to the sample chamber 320.

Multiple initially closed (indicated by an x in a box) valves 326, 331, 336, 341, and 346 on valve layer 120 are positioned on feeder channels 327, 332, 337, 342, and 347 formed on the second fluidic layer 125 between respective reagent chambers 325, 330, 335, 340, and 345. Feeder channels 327 and 347 extend to the channel 350 at valves 326 and 346. Feeder channels 332, 337, and 342 extend part way to channel 350 to align with valves 331, 336, and 341. Additional feeder channels 333, 339, and 343 extend in fluidic layer 115 from channel 350 to align with respective valves 331, 336, and 341. Selected ablation or melting of the valves 326, 330, 336, 341, and 346 allows fluid to flow between the reagent chambers and the sample chamber 320 via the feeder channels and channel 350.

Similarly, multiple valves 356, 361, 366, 371, and 376 on valve layer 120 are positioned between channel 380 and the recovery chambers 355, 360, 365, 370, and 375. A plurality of feeder channels 381, 382, 383, 384, and 385 are coupled to the recovery chambers. Feeder channels 381, 382, 383, 384 are formed on first fluidic layer 115 and extend to align with respective valves 361, 366, 371, and 376. Feeder channel 385 is formed on the second fluidic layer 125 and is directly coupled to channel 380.

Valves 356, 361, 366, 371, and 376 enable fluid to flow through channel 380 on the second fluidic layer 125, but block flow to the associated recovery chambers until opened. Each of the reagent and recovery chambers may include ports indicated at 382 and 384 on chamber 325 for example for filling or venting to facilitate fluid flow.

Channel 350 is shown as an arc having a varying radius such that the sample chamber 320 is coupled to channel 350 that is radially furthest from a center of the assembly 100. Such a structure facilitates fluid flow from the reagent chambers into the sample chamber 320.

Channel 380 extends in a single arc from the sample chamber 320 sequentially toward the recovery chambers with increasing radius. This increasing radius arc structure of channel 380 facilitates filling of the recovery chambers extending along the arc, with chamber 375 positioned furthest from the sample chamber 320 and also furthest from the axis or center of the assembly 100.

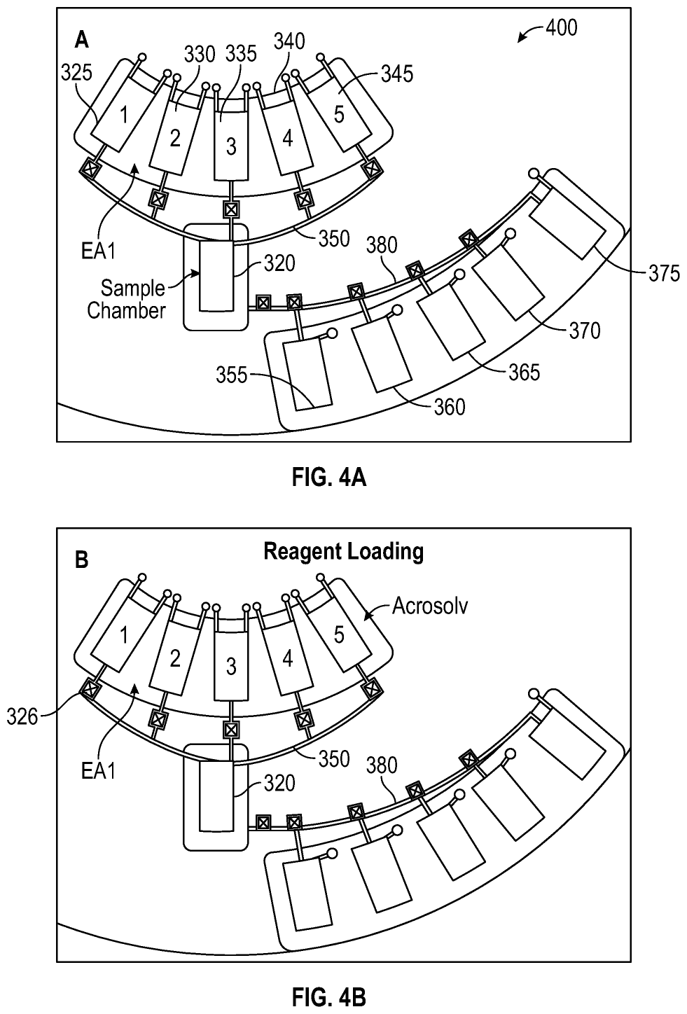

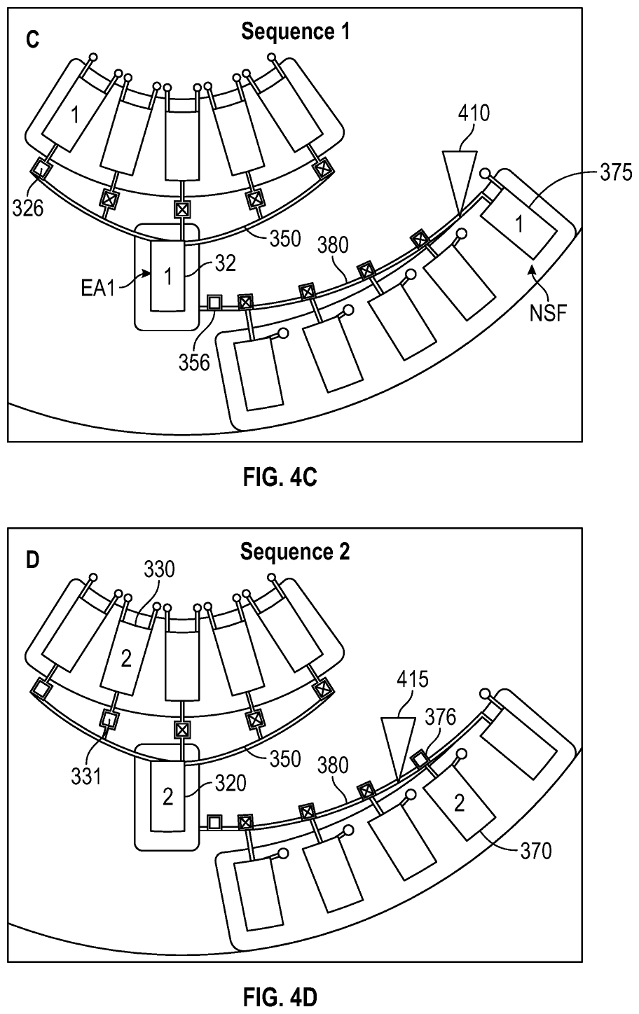

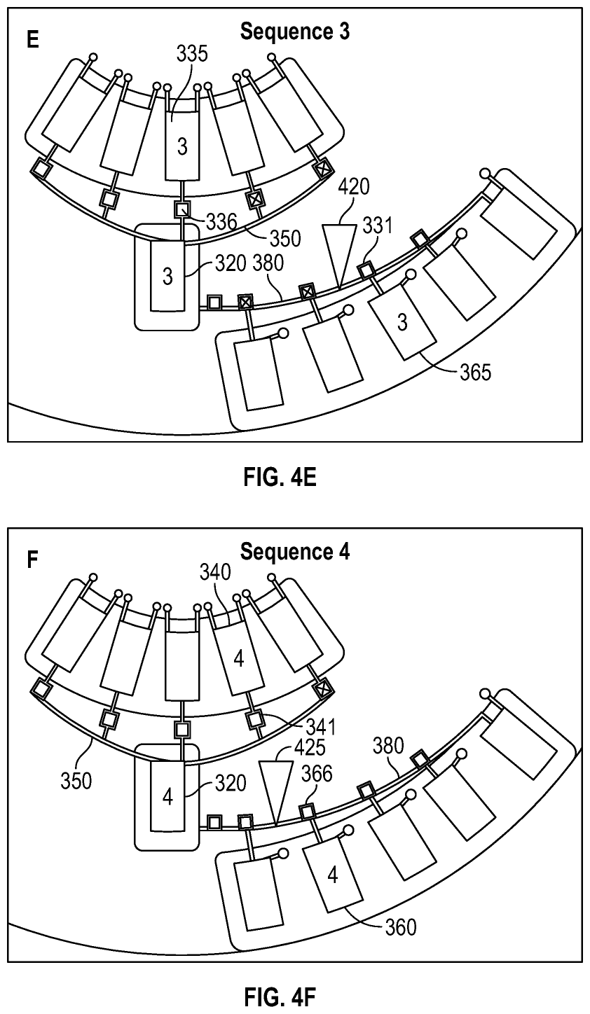

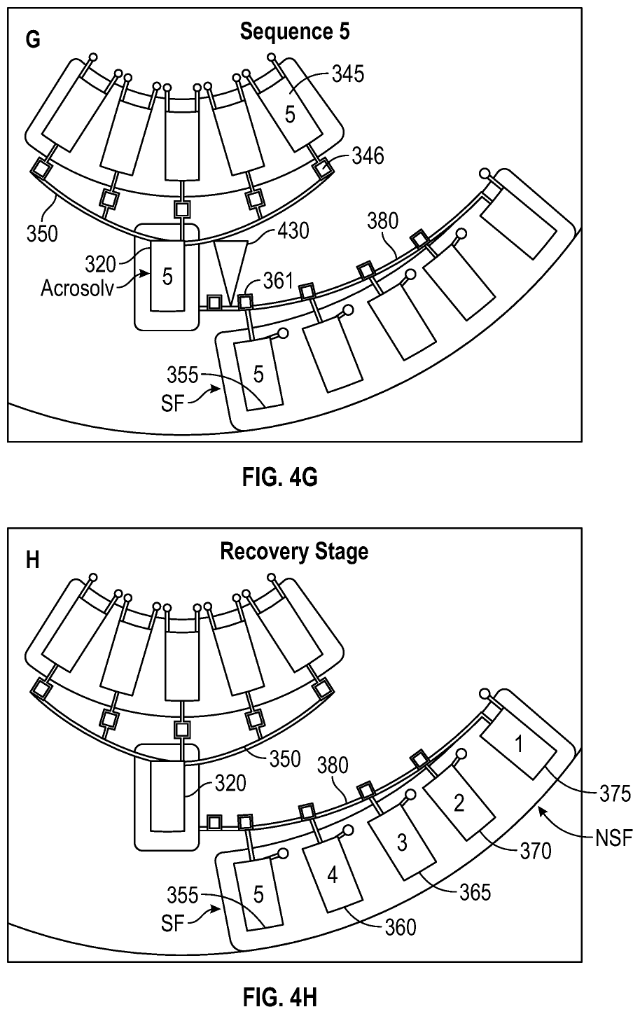

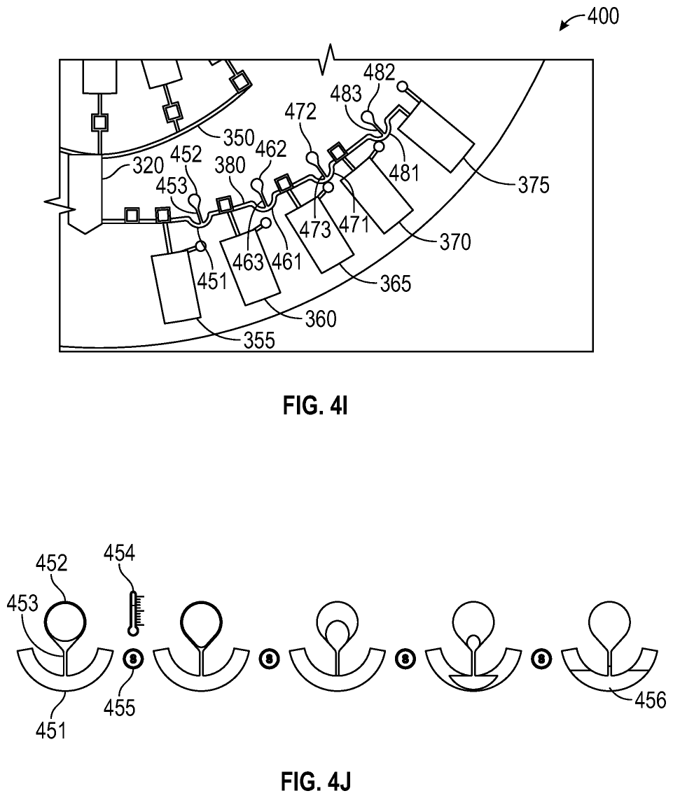

FIGS. 4A, 4B, 4C, 4D, 4E, 4F, FIGS. 4G and 4H illustrate process workflow generally at 400 for performing DE. Stepwise valve opening and closing events control fluid flow from reagent chambers into the sample chamber and subsequent recovery in the recovery chambers. Spinning is used to move fluid between chambers. Most centrifugal microfluidic systems operate at modest rotational frequencies of 10-50 Hz (600-3,000 rpm). In one example, the contents of the sample chamber 320 positioned 6 cm from the center of rotation of the assembly 100 (some space is required for downstream recovery chambers) and rotated at 3,000 rpm to provide a sufficient fluid movement force of about 604*g. Positioning the chambers radially and varying the diameters of the layers may result in different rotation speeds being used.

FIG. 4A represents a layout of domain 310 with numbered reagent chambers. Process flow 400 begins by inserting a swab cutting containing evidence into sample chamber 320 via accessory piece 140 and covering the sample chamber 320 via coverlet 150 applied to accessory piece 140. Reagents are also loaded into the reagent chambers 330, 335, 340, 345, and 350 via accessory piece 145. Coverlet 160 is applied to seal the reagent chambers. The loaded assembly 100 may then be placed into a microfluidic processing system 200 to enable lasing and spinning.

In FIG. 4B, valve 326 is opened and the NSLC is spun into the sample chamber as shown in FIG. 4C. Heating is then performed to lyse e-cells and extract DNA. In one example, heating may include exposing the sample chamber 320 to temperatures in the range of 75-95° C. for 6-10 minutes. The temperatures and ranges used herein are used to mimic standard DE processes and may be varied in different examples.

In FIG. 4C, valve 356 is opened and extracted DNA from the sample chamber 320 is spun into recovery chamber 375. Once the DNA is spun into chamber 375, the channel 380 is closed between chambers 375 and 370 as indicated at 410, which represents laser light used to melt material in the valve layer to block the channel 380 at the point shown. Closing the channel 380 at this point sequesters NSF into chamber 375.

FIG. 4D represents the opening of valve 331 and spinning of wash, such as water from chamber 330 into the sample chamber 320. This is followed by opening of valve 376 and spinning to transfer the wash into chamber 370. Note that the downstream chamber 375 is blocked via channel closure occurring at 410, ensuring the wash is captured in chamber 370. Follow transfer of the wash, the channel 380 is closed upstream of chamber 370 as indicated at 415 to sequester the wash into chamber 370.

FIG. 4E represents the opening of valve 336 and spinning of wash, such as water from chamber 335 into the sample chamber 320. This is followed by opening of valve 371 and spinning to transfer the wash into chamber 365. Note that the downstream chamber 370 is blocked via channel closure occurring at 415, ensuring the wash is captured in chamber 365. Following transfer of the wash, the channel 380 is closed upstream of chamber 365 as indicated at 420 to sequester the wash into chamber 365.

FIG. 4F represents the opening of valve 341 and spinning of wash, such as water from chamber 340 into the sample chamber 320. This is followed by opening of valve 366 and spinning to transfer the wash into chamber 360. Note that the downstream chamber 370 is blocked via channel closure occurring at 420, ensuring the wash is captured in chamber 360. Following transfer of the wash, the channel 380 is closed upstream of chamber 360 as indicated at 425 to sequester the wash into chamber 360. The number of washes may be varied in further examples by either not opening valves or modifying the assembly 100 to include more or fewer chambers designated for washing reagents.

FIG. 4G represents the opening of valve 346 and spinning of SLC from chamber 345 into the sample chamber 320. The Sample chamber 320 is incubated by heating to lyse sperm cells and extract DNA. This is followed by opening of valve 366 and spinning to transfer the DNA comprising SF into chamber 355. Note that the downstream chamber 365 is blocked via channel closure occurring at 425, ensuring the SF is captured in chamber 355. Following transfer of the SLC and SF, the channel 380 is closed upstream of chamber 355 as indicated at 430 to sequester the SF into chamber 360.

FIG. 4H represents a recovery stage in which individual fractions may be manually recovered from chamber 355 via coverlet 160 for performing PCR and further comparison to other DNA to find a match. Fluids from the other recovery chambers may also be recovered and used for desired downstream processes for data analysis and interpretation.

FIGS. 4I and 4J represent an alternative valving method for closing valves. Channel 380 is shown as including sets of “U” shaped closeable channels 451, 461, 471, and 481 disposed in channel 380 at the same positions channel closures occurred at 410, 420, 430, and 430 by melting PeT polyethylene terephthalate (PET) layer (either inherently black, or clear PeT coated with a black substance, e.g., toner) between two distinct fluidic layers 115, 125 such as in valve layer 120.

To create a valve closing structure, the closeable channels 451, 461, 471, and 481 are each paired with corresponding chambers 452, 462, 472,482 and channels 453. 463, 473, 483. In a resting state, each chamber 452, 462, 472,482 contains a plug of meltable material, such as wax. Ferrowax may be used in one example. The valve closing structures may easily be incorporated in the manufacturing process through a simple ‘pick and place’ tool.

FIG. 4J illustrates various states of the valve closing structure during valve closing. The wax material would remain in solid form until heated to the appropriate temperatures at 454 so as to induce melting followed by a spin step 455 to force the liquid wax into the U-shaped chambers 451, 461, 471, 481 via the channels 453. 463, 473, 483. The timing between chamber heating and spin speed/time is coordinated in a manner that allows flow followed by solidification which effectively closes the channels between valves at 456. Channels 453. 463, 473, 483

[symbol]

The process chain as illustrated in FIG. 4J involves laser-irradiating the solid plug and spinning the assembly 100 to promote flow of the now liquefied wax. The ferrowax is a good choice for the meltable material as iron particle imparting a black color to the wax allows for focused irradiation from an infrared light source (red laser or bulb) to specifically and rapidly melt the wax. This allows use of the same laser that was used above to melt PeT to both open and close valves as a heat source to promote the necessary phase change in the wax. With the use of the alternative valving method, the black PeT is not used for channel closure. Spin speeds and times that promote adequate flow of melted wax into the u-shaped channel without premature solidification and consequent clogging of intermediate channels may be empirically determined. Other embodiments for closable valving on devices fabricated from glassy polymers or other thermoplastics that are used in injection molding or hot embossing may alternatively be used.

The workflow utilizing assembly 100 forgoes the aggressive vortexing, reducing agents, and prolonged incubation times (>2 hrs.) associated with conventional DE. One example workflow implements a process utilizing two enzymes, a proteolytic enzyme cocktail (available as forensicGEM or prepGEM from MicroGEM Corp., Hamilton, NZ) and Acrosolv (MicroGEM). Much like conventional forensic DE, the first enzyme is a proteolytic enzyme that preferentially lyses e-cells and non-sperm cells. However, unlike Pro K, the proteolytic enzyme cocktail does not require the assistance of concentrated ionic or anionic detergents, which are known to adversely affect downstream PCR assays. Acrosolv, a proprietary mixture of proteases, is designed to release DNA from sperm nuclei without the need for reducing agents. Removing non-sperm cell DNA from the sample chamber and downstream architecture is performed with water or buffer washes.

To dislodge unbound or loosely bound sperm cells from evidence cuttings, conventional DE relies upon aggressive vortexing and centrifugation. Anecdotal reports suggest that this aggressive vortex-centrifugation approach fails to dislodge a significant proportion of the sperm cells. A swab-in approach is used for the assembly 100 and is intended to exploit the natural tendency to maximize sperm cell DNA recovery by retaining unbound and loosely bound sperm cells and by lysing them directly from the cutting during the last enzymatic treatment step of the workflow. Simply stated, unlike conventional DE, the swab-in approach retains and treats the cutting throughout the workflow to improve yield.

In one example, the sample provided to sample chamber 320 may be obtained from cutting away fibrous tips of sterile 6″ cotton swabs (Puritan Medical Products, Guilford, ME, U.S.A.) from the wooden shaft using a standard fine point blade (#11 X-ACTO®, Elmer's Products, Inc., High Point, NC) or a single-edge industrial razor blade (#9 carbon steel, VWR International, LLC., Radnor, PA, U.S.A.). Blades may be pre-cleaned with 10% bleach and methanol. The sample chambers may be 6 cm from a center of rotation (CoR) of the assembly 100.

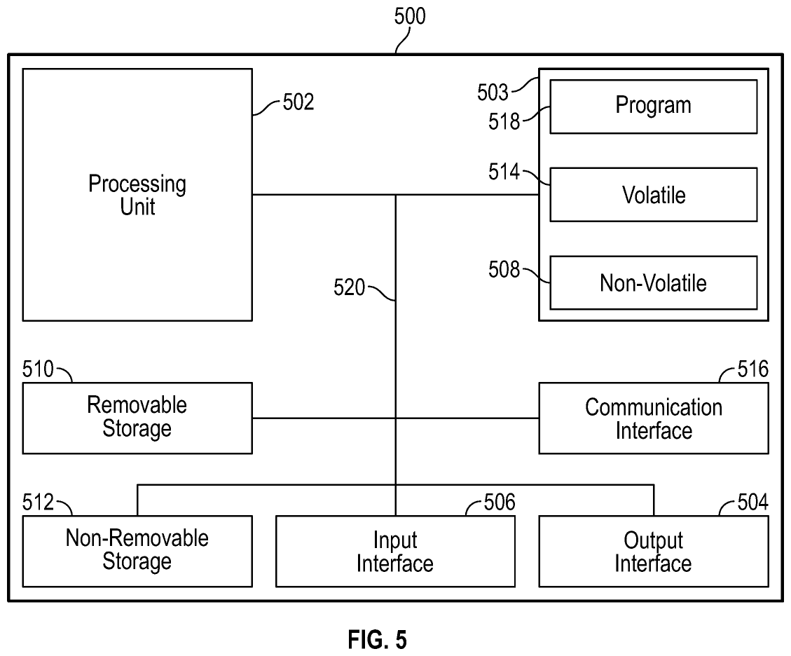

FIG. 5 is a block schematic diagram of a computer system 500 to operate the system, control processes performed on the assembly 100, and for controlling workflow as well as to actuate the laser for opening and controlling the laser controlled valves or other valves, and for performing methods and algorithms according to example embodiments. All components need not be used in various embodiments.

One example computing device in the form of a computer 500 may include a processing unit 502, memory 503, removable storage 510, and non-removable storage 512. Although the example computing device is illustrated and described as computer 500, the computing device may be in different forms in different embodiments. For example, the computing device may instead be a smartphone, a tablet, smartwatch, smart storage device (SSD), or other computing device including the same or similar elements as illustrated and described with regard to FIG. 5. Devices, such as smartphones, tablets, and smartwatches, are generally collectively referred to as mobile devices or user equipment.

Although the various data storage elements are illustrated as part of the computer 500, the storage may also or alternatively include cloud-based storage accessible via a network, such as the Internet or server-based storage. Note also that an SSD may include a processor on which the parser may be run, allowing transfer of parsed, filtered data through I/O channels between the SSD and main memory.

Memory 503 may include volatile memory 514 and non-volatile memory 508. Computer 500 may include—or have access to a computing environment that includes—a variety of computer-readable media, such as volatile memory 514 and non-volatile memory 508, removable storage 510 and non-removable storage 512. Computer storage includes random access memory (RAM), read only memory (ROM), erasable programmable read-only memory (EPROM) or electrically erasable programmable read-only memory (EEPROM), flash memory or other memory technologies, compact disc read-only memory (CD ROM), Digital Versatile Disks (DVD) or other optical disk storage, magnetic cassettes, magnetic tape, magnetic disk storage or other magnetic storage devices, or any other medium capable of storing computer-readable instructions.

Computer 500 may include or have access to a computing environment that includes input interface 506, output interface 504, and a communication interface 516. Output interface 504 may include a display device, such as a touchscreen, that also may serve as an input device. The input interface 506 may include one or more of a touchscreen, touchpad, mouse, keyboard, camera, one or more device-specific buttons, one or more sensors integrated within or coupled via wired or wireless data connections to the computer 500, and other input devices. The computer may operate in a networked environment using a communication connection to connect to one or more remote computers, such as database servers. The remote computer may include a personal computer (PC), server, router, network PC, a peer device or other common data flow network switch, or the like. The communication connection may include a Local Area Network (LAN), a Wide Area Network (WAN), cellular, Wi-Fi, Bluetooth, or other networks. According to one embodiment, the various components of computer 500 are connected with a system bus 520.

Computer-readable instructions stored on a computer-readable medium are executable by the processing unit 502 of the computer 500, such as a program 518. The program 518 in some embodiments comprises software to implement one or more methods described herein. A hard drive, CD-ROM, and RAM are some examples of articles including a non-transitory computer-readable medium such as a storage device. The terms computer-readable medium, machine readable medium, and storage device do not include carrier waves or signals to the extent carrier waves and signals are deemed too transitory. Storage can also include networked storage, such as a storage area network (SAN). Computer program 518 along with the workspace manager 522 may be used to cause processing unit 502 to perform one or more methods or algorithms described herein.

The functions or algorithms described herein may be implemented in software in one embodiment. The software may consist of computer executable instructions stored on computer readable media or computer readable storage device such as one or more non-transitory memories or other type of hardware-based storage devices, either local or networked. Further, such functions correspond to modules, which may be software, hardware, firmware or any combination thereof. Multiple functions may be performed in one or more modules as desired, and the embodiments described are merely examples. The software may be executed on a digital signal processor, ASIC, microprocessor, or other type of processor operating on a computer system, such as a personal computer, server or other computer system, turning such computer system into a specifically programmed machine.

The functionality can be configured to perform an operation using, for instance, software, hardware, firmware, or the like. For example, the phrase “configured to” can refer to a logic circuit structure of a hardware element that is to implement the associated functionality. The phrase “configured to” can also refer to a logic circuit structure of a hardware element that is to implement the coding design of associated functionality of firmware or software. The term “module” refers to a structural element that can be implemented using any suitable hardware (e.g., a processor, among others), software (e.g., an application, among others), firmware, or any combination of hardware, software, and firmware. The term, “logic” encompasses any functionality for performing a task. For instance, each operation illustrated in the flowcharts corresponds to logic for performing that operation. An operation can be performed using, software, hardware, firmware, or the like. The terms, “component,” “system,” and the like may refer to computer-related entities, hardware, and software in execution, firmware, or combination thereof. A component may be a process running on a processor, an object, an executable, a program, a function, a subroutine, a computer, or a combination of software and hardware. The term, “processor,” may refer to a hardware component, such as a processing unit of a computer system.

Furthermore, the claimed subject matter may be implemented as a method, apparatus, or article of manufacture using standard programming and engineering techniques to produce software, firmware, hardware, or any combination thereof to control a computing device to implement the disclosed subject matter. The term, “article of manufacture,” as used herein is intended to encompass a computer program accessible from any computer-readable storage device or media. Computer-readable storage media can include, but are not limited to, magnetic storage devices, e.g., hard disk, floppy disk, magnetic strips, optical disk, compact disk (CD), digital versatile disk (DVD), smart cards, flash memory devices, among others. In contrast, computer-readable media, i.e., not storage media, may additionally include communication media such as transmission media for wireless signals and the like.

Although a few embodiments have been described in detail above, other modifications are possible. For example, the logic flows depicted in the figures do not require the particular order shown, or sequential order, to achieve desirable results. Other steps may be provided, or steps may be eliminated, from the described flows, and other components may be added to, or removed from, the described systems. Other embodiments may be within the scope of the following statements.

1. A microfluidic differential extraction device comprising:
a first fluidic layer;
a second fluidic layer;
a valving layer disposed between the first and second fluidic layers, the valving layer having multiple valves;
a plurality of reagent chambers extending through the layers;
a sample chamber extending through the layers;
a plurality of recovery chambers extending through the layers; and
microfluidic channels formed in the first fluidic layer and the second fluidic layer that selectively couple the reagent chambers to the sample chamber and the sample chamber to the recovery chamber in response to actuation of the valves.

2. The device of claim 1 and further comprising top and bottom layers sandwiching the first and second fluidic layers and valving layers.

3. The device of claim 2 and further comprising accessory pieces to couple to the top layer and extend the reagent, sample, and recovery chamber volume.

4. The device of claim 3 and further comprising coverlets to bond to the accessory pieces to seal the reagent, sample, and recovery chambers.

5. The device of claim 1 wherein the microfluidic channels include main channels formed in arcs of increasing radius from a center axis of the device.

6. The device of claim 1 wherein the layers are disc shaped about a central axis and wherein the chambers and microfluidic channels are configured to move fluid in response to spinning of the layers about the central axis.

7. The device of claim 1 and further comprising means for moving fluid between the chambers.

8. The device of claim 1 and further comprising multiple separate sets of chambers, microfluidic channels, and valves disposed in the first and second fluidic and valve layers.

9. The device of claim 1 wherein the fluidic layers are transparent and the valves in the valve layer are actuatable via heat provided by a laser.

10. The device of claim 1 wherein the valves in the valve layer are disposed between corresponding microfluidic channels extending in the first and second fluidic layers from the reagent chambers and recovery chambers.

11. The device of claim 1 wherein the valves between the reagent chambers and the sample chamber are disposed to control fluid flow through feeder channels formed in the first and second fluidic layers.

12. The device of claim 1 wherein the reagent chambers and recovery chambers match in number and wherein the valves are controllable to sequentially fill the sample chamber and empty contents of the sample chamber into a corresponding recovery chamber.

13. The device of claim 12 wherein the microchannels are actuatable to close to sequentially sequester contents in the recovery chambers.

14. A method of performing differential extraction of DNA from a sample, the method comprising:
placing the sample in a sample chamber;
filling reagent chambers with reagent; and
iteratively, for each reagent chamber and a corresponding recovery chamber:
transferring reagent from the reagent chamber to the sample chamber to react with the sample; and
transferring material from the sample chamber to the corresponding recovery chamber.

15. A system comprising:
an assembly including first and second fluidic layers sandwiching a valve layer and having multiple chambers and microfluidic channels positioned to sequentially control fluid flow between multiple reagent chambers and a sample chamber and between the sample chamber and multiple recovery chambers;
a laser positioned to project light onto the valve layer to actuate multiple valves disposed between the microfluidic channels;
a motor coupled to the assembly to rotate the assembly to move fluid within the assembly; and
a controller coupled to the laser and the motor to control movement of the fluid and provide centrifugal force to facilitate separation of DNA in the fluid.

16. The method of claim 14 wherein transferring reagent or material comprises spinning the layers about a central axis of the layers.

17. The method of claim 14 and further comprising actuating a valve prior to transferring reagent or material.

18. The method of claim 17 wherein actuating the valve comprises heating the valve by a laser.

19. The system of claim 15 wherein the valves in the valve layer are disposed to control fluid flow through feeder channels formed in the first and second fluidic layers.

20. The system of claim 15 wherein the reagent chambers and recovery chambers match in number and wherein the valves are controllable to sequentially fill the sample chamber and empty contents of the sample chamber into a corresponding recovery chamber.
